(19) United States
(12) Patent Application Publication
(10) Pub. No.: US 2008/0171984 A1
(43) Pub. Date: Jul. 17, 2008

(54) Cannula driver and system

(76) Inventor: Peter C. Miller, Largo, FL (US)

Correspondence Address: Michael de Angeli, 60 Intrepid Lane, Jamestown, RI, 02835, US

(21) Appl. No.: 12/007,075

(22) Filed: Jan. 7, 2008

Related U.S. Application Data
(60) Provisional application No. 60/879,884, filed on Jan. 11, 2007.

(51) Int. Cl. A61M 5/00

(52) U.S. Cl. 604/117

(57) ABSTRACT

A cannula and cannula driver system comprises a tubular cannula and a driver, the driver comprising an elongated obturator shaft and a handle. A noncircular recess is formed on one of the of the cannula and the obturator shaft which receives a correspondingly-shaped projection formed about the other of the cannula and obturator shaft, so that when the obturator shaft is inserted into the tubular cannula, the projection fits into the recess, whereby torque applied to the handle of the driver is transferred to the cannula.

### CROSS-REFERENCE TO RELATED APPLICATION

This application claims priority from Provisional Application Ser. No. 60/879,884, filed Jan. 11, 2007.

### FIELD OF THE INVENTION

The invention relates to surgical devices used to facilitate access to surgical sites during endoscopic surgical procedures. More particularly, the invention relates to a cannula system for inserting cannulas.

### BACKGROUND OF THE INVENTION

Cannulas are tubular devices used to establish and maintain portals to enable elongated instruments to be used at a surgical site during endoscopic procedures. Some cannulas are threaded or ribbed to enable them to be secured in a desired position by being turned or pushed into place through the patient's body tissue. Particularly where the cannula is of a relatively flexible material, it is known to use a cannula driver, comprising a handle and a distally extending elongated obturator shaft, to assist in placement of the cannula. The tubular cannula is placed over the elongated obturator shaft, so that the shaft supports the cannula as it is inserted into the wound. The tip of the obturator shaft may be pointed to assist in placement of the cannula tip at the desired surgical site. The handle is then used to conveniently grip the assembly of the cannula and obturator shaft.

It is known to provide the handle of the cannula driver and the proximal end of the cannula with cooperating keyed structures, such that torque can be applied by the surgeon to the handle to turn the cannula while also applying force to push the cannula into position. If the cannula is provided with a screw thread at its distal tip, torque can be applied to the handle of the cannula driver to assist in securing the cannula to the tissues at the surgical site.

One known cannula driver comprises male members on either side of the shaft of the obturator, at the distal end of the handle thereof, cooperating with female recesses formed in the proximal end of the cannula.

This invention relates to an improved cannula driver system.

### SUMMARY OF THE INVENTION

According to the preferred embodiment of the invention, a noncircular recess is formed on the proximal surface of a cannula which receives a correspondingly-shaped projection formed about the base of the obturator shaft of a cannula driver, adjacent the handle, so that when the obturator shaft is inserted into the tubular cannula, the projection fits into the recess, whereby torque applied to the handle of the driver is transferred to the cannula. Alternatively, the projection can be formed on the cannula and the cooperating recess on the driver.

### BRIEF DESCRIPTION OF THE DRAWINGS

The invention will be better understood if reference is made to the accompanying drawings, in which:

FIG. 1 is a front perspective view of a cannula driver constructed in accordance with the principles of this invention;

FIG. 2 is a right end view of the cannula driver of FIG. 1;

FIG. 3 is a rear perspective view of a cannula intended to be used with the cannula driver of FIG. 1;

FIG. 4 is a left end view of the cannula of FIG. 3;

FIG. 5 is a perspective view of the cannula driver of FIG. 1 and the cannula of FIG. 3; and

FIG. 6 is a side elevation view of the components of FIG. 5 partially assembled together.

### DESCRIPTION OF THE PREFERRED EMBODIMENTS

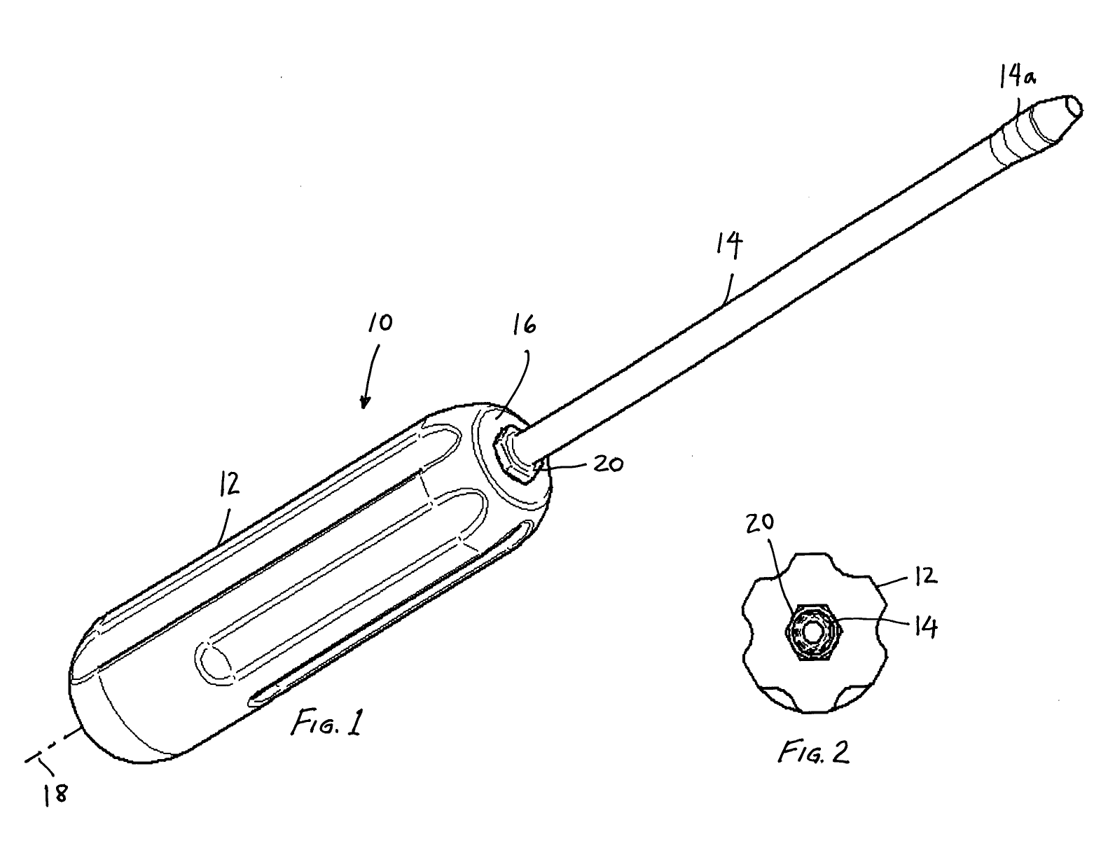

As shown in FIG. 1, the cannula driver 10 according to the invention comprises a handle 12 and an obturator shaft 14 attached to the distal end 16 of handle 12. Handle 12 and obturator shaft 14 are coaxially aligned along axis 18. Interposed between shaft 14 and distal end 16 is a distally extending, noncircular projection 20 extending around axis 18. The projection can be rectilinear, that is, have a cross-sectional shape made up of straight line segments in a plane perpendicular to the axis 18, or curvilinear in that plane, and may or may not have a consistent cross-section over its length. Preferably, the cross-section of projection 20 defines a regular polygon, so that it can be inserted into a cooperating recess (as discussed below) at a plurality of relative angular orientations. In the preferred embodiment, projection 20 is hexagonal in cross-section, as best seen in FIG. 2.

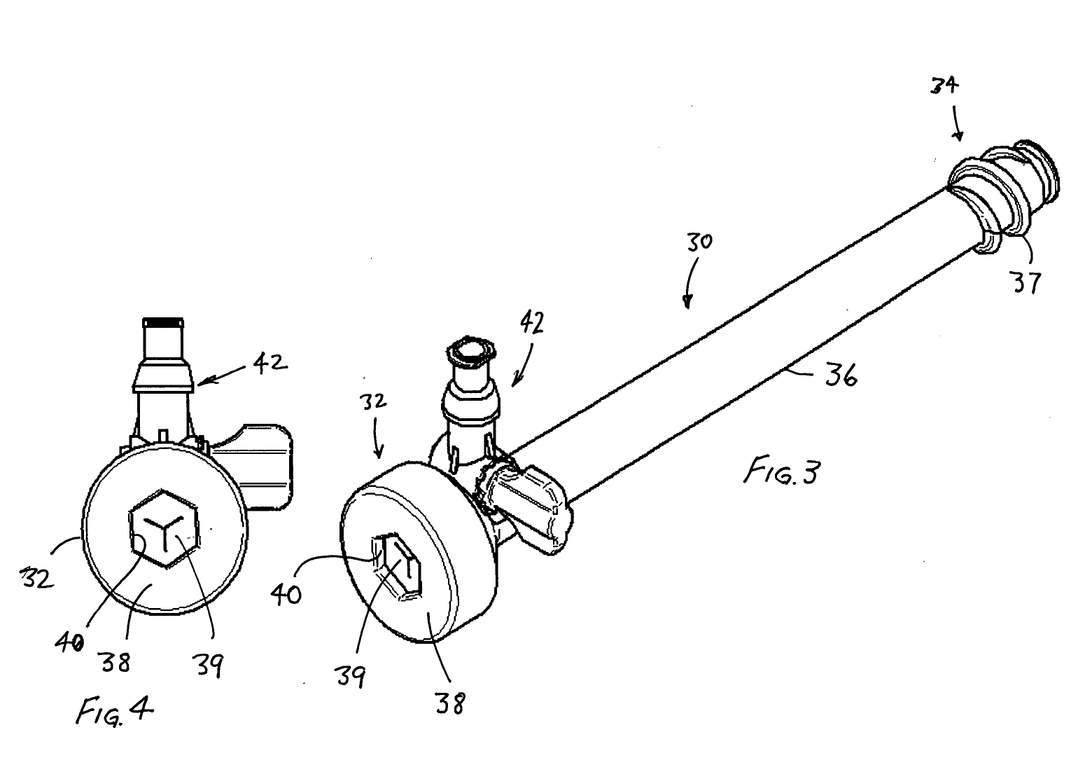

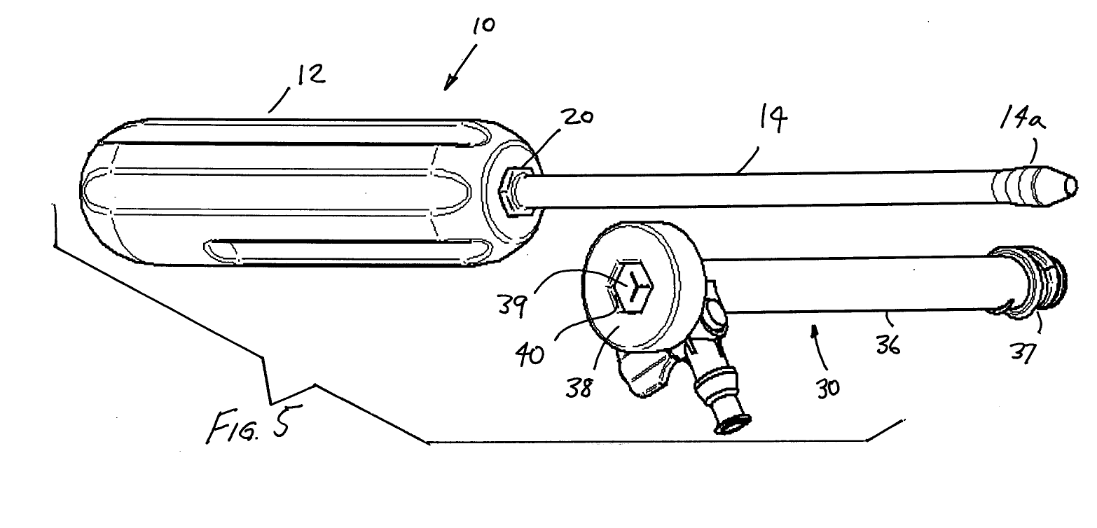

Cannula driver 10 is adapted to cooperatively engage and drive cannula 30, which is best seen in FIG. 3. Cannula 30 has a proximal end 32, a distal end 34, and an elongated tubular body 36. A valve 39 may be provided at the proximal end of the interior of tubular body 36. Conventional structure may be provided as indicated generally at 42, e.g. for the introduction of instruments, application of irrigation and/or suction, and the like. The distal tip 34 of the cannula may comprise a coarse thread 37, for securing the cannula tip to the patient's tissues at the desired site.

According to one aspect of the present invention, the proximal end 32 of cannula 30 comprises a proximally facing surface 38 that is formed to define an axially aligned, noncircular recess 40 shaped cooperatively with respect to projection 20 of cannula driver 10, so that projection 20 fits snugly within recess 40. Thus, in the preferred embodiment, recess 40 has a hexagonal profile. The axial length of projection 20 is chosen with respect to the depth of the recess so that the projection 20 fits into and engages cooperating recess 40, but does not necessarily contact the bottom of the recess 40 before the distal end 16 of handle 12 abuts the cannula's proximal surface 38. In this way, torque applied by the surgeon to handle 12 is effectively transferred to cannula 30, while axial force can be effectively applied as well, directly from the distal end 16 of the handle to the proximal mating surface 38 of the cannula.

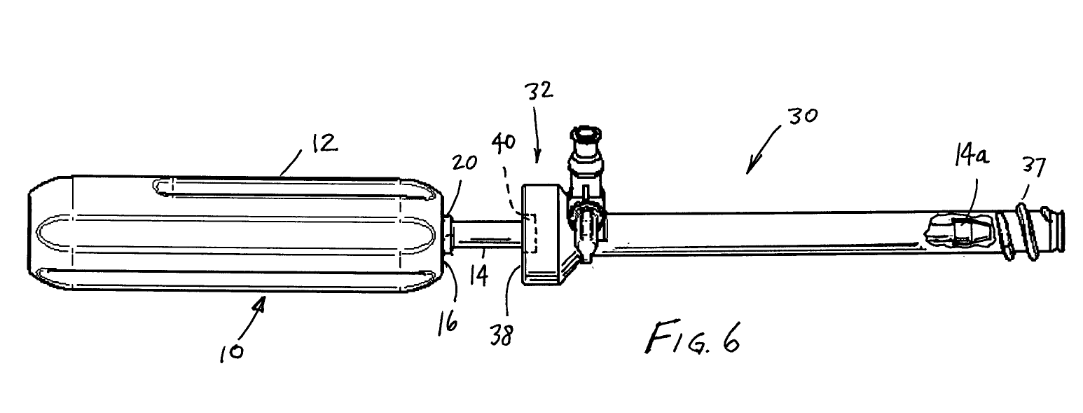

As best seen in FIGS. 5 and 6, the length of obturator shaft 14 should be sufficient to extend into and typically through the body of cannula 30, so that the tip 14a of obturator shaft 14 protrudes from the distal end of cannula 30. In this way, if the obturator shaft 14 is made of a hard, stiff material, the tip 14a of the obturator shaft 14 can be employed to guide the cannula 30 to the surgical site, while the cannula need not be made of a rigid material, but can be made of relatively soft material, for example, a translucent or transparent plastic material. In a typical embodiment, where the cannula 30 is intended for a single use, that is, is disposable, the cannula driver 10 will likewise be disposable. Both may be made principally of surgical-grade plastic materials.

As shown in FIG. 6, the cannula and cannula driver are cooperatively shaped and sized such that when the obturator shaft 14 is inserted into the proximal end 32 of cannula 30, projection 20 is inserted into cooperatively-shaped recess 40. Following complete engagement of driver 10 with cannula 30, the engagement of projection 20 with recess 40 enables a user to apply torque to handle 10, turning cannula 30 into place, while simultaneously pushing axially against the cannula with the driver.

It will be apparent to those of skill in the art that while a preferred embodiment of the invention has been disclosed, there are numerous modifications and additions that can be made thereto without departure from its essential spirit and scope. For example, the male projection 20 on cannula driver 10 and female recess 40 on cannula 30 could have many different cooperating shapes. Preferably, the shapes chosen are rectilinear, and are profiled such that the projection 20 can be inserted into the recess 40 at a number of angular orientations, simplifying use of the device. For example, the cooperating shapes could be regular polygonal shapes, e.g., equilateral triangles, squares, pentagons, octagons, or hexagons, as in the preferred embodiment shown. Typically, the distal edges of the projection 20 will be relieved slightly, to facilitate insertion of the projection into the corresponding recess 40. Further, the recess 40 could be provided on the cannula driver 10 and the projection 20 on the cannula 30.

Therefore, the above disclosure of the invention should be considered exemplary only, and not as a limitation on its scope, which is defined by the claims which follow.

What is claimed is:

1. A cannula and cannula driving system, comprising:
a cannula comprising:
an elongated tubular body having an axis, a proximal end and a distal end;
a proximally facing surface at the proximal end of said cannula, said surface situated transversely to said axis; and
a cannula driver comprising:
an elongated obturator shaft having an axis, a proximal end and a distal end, said obturator shaft being received within said tubular body of said cannula; and
a handle having an axis, a proximal end, and a distal end, said distal end of said handle being secured to the proximal end of said elongated obturator shaft, said handle having a distally facing surface at its distal end,
one of said cannula and said driver defining a noncircularly profiled, coaxially aligned recess and the other of said cannula and said driver comprising a correspondingly noncircularly profiled, coaxially aligned projection having a cross-sectional shape corresponding to the shape of said recess, so that said projection can be received in said recess,
whereby torque applied by a user to said handle of said cannula driver is transmitted by the interfitting projection and recess to said proximal end of said cannula, so as to rotate said cannula.

2. The cannula and cannula driving system of claim 1, wherein said projection and said recess are shaped so as to define cooperating rectilinear cross-sectional shapes.

3. The cannula and cannula driving system of claim 2, wherein said projection and said recess are shaped so as to define cross-sectional shapes of regular polygons, such that said projection can be received within said recess at radially spaced respective positions for transmission of torque therebetween.

4. The cannula and cannula driving system of claim 3, wherein said projection is hexagonal in cross-section and said recess is cooperatively hexagonal.

5. The cannula and cannula driving system of claim 1, wherein the length of said obturator shaft, the length of said tubular cannula, the length of said projection, and the depth of said recess are chosen cooperatively such that when the distally facing surface of the handle abuts the proximally facing surface at the proximal end of said cannula, the projection extends into but does not reach the bottom of the recess.

6. The cannula and cannula driving system of claim 5, wherein when the distally facing surface of the handle abuts the proximally facing surface at the proximal end of said cannula, the distal tip of the obturator shaft protrudes from the distal tip of the cannula.

7. The cannula and cannula driving system of claim 1, wherein the projection is formed on the cannula driver and the cooperating recess on the cannula.
